(19) United States
(12) Patent Application Publication
(10) Pub. No.: US 2006/0094913 A1
(43) Pub. Date: May 4, 2006

(54) Ion pair amphiphiles as hydrate inhibitors

(76) Inventor: Paul Anthony Spratt, Missouri City, TX (US)

Correspondence Address: NALCO COMPANY, 1601 W. DIEHL ROAD, NAPERVILLE, IL, 60563-1198, US

(21) Appl. No.: 10/981,230

(22) Filed: Nov. 4, 2004

(51) Int. Cl. C07C 9/00

(52) U.S. Cl. 585/15

(57) ABSTRACT

A method of inhibiting hydrates in a fluid comprising water and gas comprising adding to the fluid an effective hydrate-inhibiting amount of one or more ion-pair amphiphiles, wherein the ion-pair amphiphiles are composed of one or more cationic amphiphiles and one or more anionic amphiphiles.

### TECHNICAL FIELD

This invention relates to inhibiting the formation, growth and aggregation of hydrate particles in fluids containing hydrocarbon gas and water, particularly in the production and transport of natural gas, petroleum gas or other gases.

### BACKGROUND OF THE INVENTION

The formation of clathrate hydrates occurs when water and low molecular weight compounds such as carbon dioxide, hydrogen sulfide, methane, ethane, propane, butane and iso-butane are in contact at low temperatures and increased pressures. Under these conditions, the clathrate hydrates form a cage-like crystalline structure that incorporates guest molecules such as hydrate forming hydrocarbons and gases. While these crystalline cages are small initially (1-3 nm), they are able to agglomerate and increase in size rapidly. The clathrate hydrate crystals, when allowed to form and grow inside a conduit such as a pipeline, tend to block or even damage the conduit.

The petroleum industry gives particular attention to clathrate hydrates because the conditions that are needed to form the blockages are prevalent under normal operational conditions. There are many instances where hydrate blockages have halted the production of gas, condensate, and oil. Obviously, the monetary consequences for each of these instances are amplified when considering the volumes of production in deepwater applications where tens of thousands of barrels of oil are routinely produced daily and the shut-ins can take months to remedy. Additionally, restarting a shutdown facility, particularly an offshore production or transportation facility, is extremely difficult because of the significant amounts of time, energy, and materials, as well as the various engineering implementations that are often required to remove a hydrate blockage under safe conditions.

A number of methods have been suggested to prevent blockages such as thermodynamic hydrate inhibitors (THI), kinetic hydrate inhibitors (KHI) and anti-agglomerates (AA). The amount of chemical needed to prevent blockages varies widely depending upon the type of inhibitor. Thermodynamic hydrate inhibitors are typically used at very high concentrations, while KHI's and AA's are used at much lower concentrations and are typically termed low dose hydrate inhibitors (LDHI).

Thermodynamic inhibitors decrease the equilibrium temperature of hydrate formation and change thermodynamic properties. This has the effect of reducing the amount of subcooling in the system. Subcooling is defined as the differential in temperature between where hydrates can be formed and the actual operating conditions. For example, thermodynamics show that hydrates will form at 70° F. at a certain pressure, but the operating temperature is 40° F. This would give a subcooling of 30° F. A thermodynamic inhibitor would reduce the amount of subcooling when added. Thermodynamic inhibitors often have to be added in substantial amounts, typically in the order of several tens of percent by weight of the water present, in order to be effective. Common thermodynamic inhibitors are methanol, ethanol, and glycol as well as some inorganic salts.

Commonly it is accepted that the KHI interferes with the growth of the clathrate hydrate crystal, thus preventing the formation of the hydrates. Unfortunately, there are several limitations that have been discovered with the use of KHI's such as subcooling, unfavorable interactions with other chemicals, dosage levels, and expense of the commercial polymers used.

While KHI's prevent the formation of hydrate crystals by disrupting the crystal growth, the AA's allow the crystal to form and then disperse the crystal. It is commonly accepted that AA's act as dispersants of the hydrate crystals into the hydrocarbon phase, and therefore have a limitation that the liquid hydrocarbon phase must be present. Typically the liquid hydrocarbon to water ratio should be no greater then one to one to ensure that there is enough hydrocarbon to contain the dispersed hydrate crystals. Unfortunately, this limitation reduces the opportunity in the oilfield as many wells increase the amount of water produced very rapidly after the water breakthrough is observed.

Accordingly, there is an ongoing need for new and effective hydrate inhibitors.

### SUMMARY OF THE INVENTION

This invention is a method of inhibiting hydrates in a fluid comprising water, gas and optionally liquid hydrocarbon comprising treating the fluid with an effective hydrate-inhibiting amount of one or more ion-pair amphiphiles, wherein the ion-pair amphiphiles are composed of one or more cationic amphiphiles and one or more anionic amphiphiles.

The ion-pair amphiphiles of this invention effectively prevent the formation and deposition of large hydrate agglomerates in crude, gas condensate and other fuel oils, thereby improving their flow properties. The ion-pair amphiphiles possess excellent hydrate inhibition characteristics under high water cut, high subcooling and low salinity conditions.

### DETAILED DESCRIPTION OF THE INVENTION

The ion-pair amphiphiles of this invention are formed by ionic bonding of cationic and anionic amphiphiles to form a structure of formula (I).

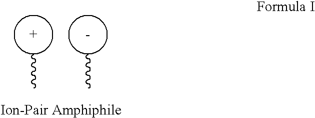

“Cationic amphiphile” means an ionic compound comprising a hydrophobic hydrocarbon portion and a hydrophilic portion capable of supporting a positive charge in aqueous solution when combined with an anionic amphiphile as defined herein.

“Anionic amphiphile” means an ionic compound comprising a hydrophobic hydrocarbon portion and a hydrophilic portion capable of supporting a negative charge in aqueous solution when combined with an anionic amphiphile as defined herein.

As used herein, “alkenyl” means a monovalent group derived from a straight or branched hydrocarbon containing at least one carbon-carbon double bond by the removal of a single hydrogen atom. Representative alkenyl groups ethenyl, propenyl, include 6-octadecenyl (oleyl, C₁₈), 9,11,13-octadecatrienyl (C₁₈), 12-hydroxy-9-octadecenyl (C₁₈), 5,8,11,14-eicosatetraenyl (C₂₀), eicosenyl (C₂₀), heneicosenyl (C₂₁), 13-docosenyl (erucyl, C₂₂), tetracosenyl (C₂₄), pentacosenyl (C₂₅), 14-methyl-11-eicosenyl, 2-hydroxy-18-oxa-19-methyl-4-eicosenyl, and the like.

“Alkoxy” means a C₁-C₄ alkyl group attached to the parent molecular moiety through an oxygen atom. Representative alkoxy groups include methoxy, ethoxy, propoxy, butoxy, and the like. Methoxy and ethoxy are preferred.

“Alkyl” means a monovalent group derived from a straight or branched chain saturated hydrocarbon by the removal of a single hydrogen atom. Representative alkyl groups include methyl, ethyl, n- and iso-propyl, n-, sec-, iso- and tert-butyl, eicosanyl (C₂₀), heneicosanyl (C₂₁); docosyl (behenyl, C₂₂); tricosanyl (C₂₃); tetracosanyl (C₂₄); pentacosyl (C₂₅), 3-, 7-, and 13-methylhexadecanyl, and the like.

“Alkylene” means a divalent group derived from a straight or branched chain saturated hydrocarbon by the removal of two hydrogen atoms, for example methylene, 1,2-ethylene, 1,1-ethylene, 1,3-propylene, 2,2-dimethylpropylene, and the like.

“Aryl” means substituted and unsubstituted aromatic carbocyclic radicals and substituted and unsubstituted heterocyclic having from 5 to about 14 ring atoms. Representative aryl include phenyl naphthyl, phenanthryl, anthracyl, pyridyl, furyl, pyrrolyl, quinolyl, thienyl, thiazolyl, pyrimidyl, indolyl, and the like. The aryl is optionally substituted with one or more groups selected from hydroxy, halogen, C₁-C₄ alkyl and C₁-C₄ alkoxy.

“Arylalkyl” means an aryl group attached to the parent molecular moiety through an alkylene group. The number of carbon atoms in the aryl group and the alkylene group is selected such that there is a total of about 6 to about 18 carbon atoms in the arylalkyl group. A preferred arylalkyl group is benzyl.

“Halo” and “halogen” mean chlorine, fluorine, bromine and iodine.

“Thermodynamic inhibitor” means a compound that decreases the equilibrium temperature of hydrate formation and change thermodynamic properties. Representative thermodynamic inhibitors include methanol, ethanol, isopropanol, isobutanol, sec-butanol, ethylene glycol, propylene glycol, and the like.

The ion pair amphiphiles of this invention are prepared by mixing an approximately equimolar amount (about 1.0 to about 1.3 molar equivalents) of one or more cationic amphiphiles with one or more anionic amphiphiles without solvent, in aqueous or non-aqueous solvents, in a mixture of aqueous and non-aqueous solvents or in the fluid being treated. The amphiphiles can be charged prior to mixing as in the case of a quaternary ammonium ion or can simply be a neutral compound that becomes charged upon introduction to the counter amphiphile, for example in the case of an amine being added to a carboxylic acid. Additionally, this can occur when the amphiphile is placed in a particular solvent as when an amine is placed in an aqueous solvent with a pH below 9 or a carboxylic acid is placed in an aqueous solvent above pH 4.

It should be noted that mixing compounds such as an amine with a carboxylic acid results in formation of a quaternary ammonium salt in an exothermic reaction. Typically it would be expected that the salt formation is very rapid, on the order of a few minutes, for liquid amphiphiles or amphiphiles that are in solution. Reaction of solid amphiphiles takes slightly longer, but would still be on the order of a few hours and likely less under most circumstances. Other then the case of salt formation, heating or cooling should not factor into the formulation of the ion pair amphiphiles.

Aqueous solvents that can suitably used in the preparation of the ion-pair amphiphiles of this invention include water, deionized water, brine, seawater, and the like.

Non aqueous solvents including aromatics such as toluene, xylene, heavy aromatic naphtha, and the like, esters such as fatty acid methyl esters, aliphatics such as pentane, hexanes, heptane, diesel fuel, and the like and glycols such as ethylene glycol and propylene glycol can suitably be used when one of the amphiphiles is in the charged state prior to addition, as in the case of a quaternary ammonium compound. In this case, if an amphiphile containing a carboxylic acid is added to the formulation in an alcohol such as methanol, it is likely that the protons would dissociate to form the anionic carboxylate anion to counter the quaternary cation.

Formulation of a particular ion pair amphiphile depends upon the application of the amphiphile and any additional treatments that will be used in conjunction with the hydrate inhibitor. For example, if the hydrate inhibitor will be injected with a paraffin inhibitor that is typically only formulated in hydrophobic solvents such as diesel, heavy aromatic naphtha, fatty acid methyl esters, xylene, toluene, and the like, the ion pair amphiphiles can also be formulated in a hydrophobic solvent to ensure that the risk of incompatibility is minimized. Alternatively, if the hydrate inhibitor will be injected with a water soluble corrosion inhibitor or scale inhibitor, a polar solvent such as methanol, ethanol, isopropanol, 2-butoxyethanol, ethylene glycol, propylene glycol, and the like, can be used.

Accordingly, in an aspect, this invention is a composition comprising one or more ion-pair amphiphiles and one or more non aqueous solvents.

In another aspect, the non-aqueous solvents are selected from the group consisting of aromatics, alcohols, esters, aliphatics, glycols, and mixtures thereof.

In another aspect, the non-aqueous solvents are selected from the group consisting of diesel, heavy aromatic naphtha, fatty acid methyl esters, xylene, toluene, and mixtures thereof.

In another aspect, the non-aqueous solvents are selected from the group consisting of methanol, ethanol, isopropanol, 2-butoxyethanol, ethylene glycol and propylene glycol and mixtures thereof.

In another aspect, this invention is a composition comprising one or more ion-pair amphiphiles in a mixture of one or more aqueous solvents and one or more non-aqueous solvents.

In another aspect, this invention is a composition comprising one or more ion-pair amphiphiles and one or more aqueous solvents, wherein the aqueous solvents are selected from brine and seawater.

In another aspect, the cationic amphiphiles are selected from the group consisting of compounds of formula 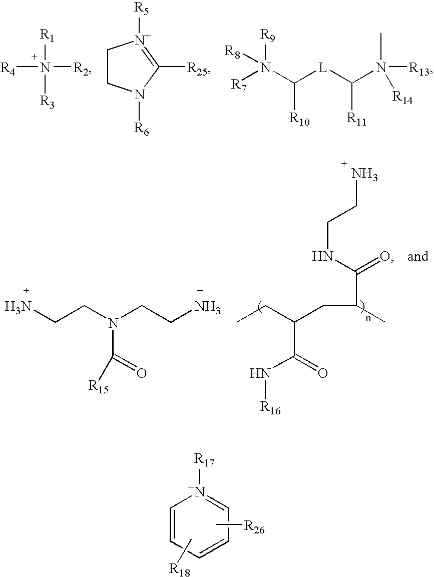
wherein R₁, R₅, R₇, R₈, R₁₂, R₁₃ and R₁₇ are independently selected from C₁-C₄ alkyl; R₂, R₉ and R₁₄ are independently selected from C₁-C₄ alkyl and arylalkyl; R₄ is C₁-C₄ alkyl, C₅-C₂₅ alkyl or C₅-C₂₅ alkenyl; R₃, R₆, R₁₀, R₁₁, R₁₅, R₁₆ and R₁₈ are independently selected from C₅-C₂₅ alkyl and C₅-C₂₅ alkenyl; R₂₅ and R₂₆ are independently selected from H, C₁-C₂₅ alkyl and C₂-C₂₅ alkenyl; L is absent, C₁-C₅ alkylene or a group of formula —CH₂CH(OH)CH₂—; and n is 1 to about 1,000.

In another aspect, the cationic amphiphiles are selected from the group consisting of compounds of formula 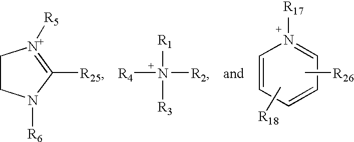
wherein R₁, R₅ and R₁₇ are independently selected from C₁-C₄ alkyl; R₂ is C₁-C₄ alkyl or arylalkyl; R₄ is C₁-C₄ alkyl, C₅-C₂₅ alkyl or C₅-C₂₅ alkenyl; R₃, R₆ and R₁₈ are independently selected from C₅-C₂₅ alkyl and C₅-C₂₅ alkenyl; and R₂₅ and R₂₆ are independently selected from H, C₁-C₂₅ alkyl and C₂-C₂₅ alkenyl.

In another aspect, the anionic amphiphile is selected from the group consisting of compounds of formula 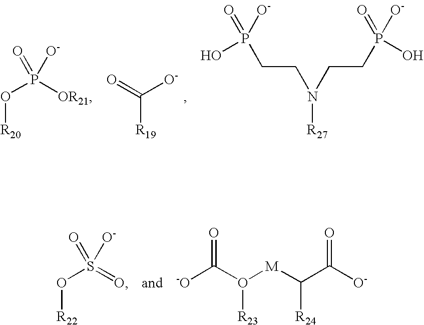
wherein R₁₉, R₂₀, R₂₂, R₂₃, R₂₇ and R₂₄ are independently selected from C₅-C₂₅ alkyl, C₅-C₂₅ alkenyl; R₂₁ is H, C₁-C₄ alkyl or arylalkyl; and M is absent or a group of formula C₁-C₅ alkylene or a group of formula —CH₂CH(OH)CH₂—.

In another aspect, the cationic amphiphiles are selected from the group consisting of compounds of formula 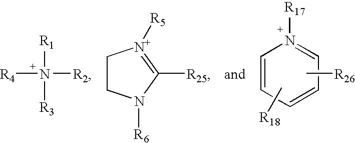
wherein R₁, R₅ and R₁₇ are independently selected from C₁-C₄ alkyl; R₂ is C₁-C₄ alkyl or arylalkyl; R₄ is C₁-C₄ alkyl, C₅-C₂₅ alkyl or C₅-C₂₅ alkenyl; R₃, R₆ and R₁₈ are independently selected from C₅-C₂₅ alkyl and C₅-C₂₅ alkenyl; and R₂₅ and R₂₆ are independently selected from H, C₁-C₂₅ alkyl and C₂-C₂₅ alkenyl and the anionic amphiphiles are selected from the group consisting of compounds of formula 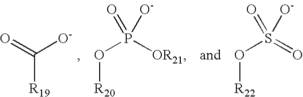
wherein R₁₉, R₂₀ and R₂₂ are independently selected from C₅-C₂₅ alkyl, C₅-C₂₅ alkenyl; and R₂₁ is H, C₁-C₄ alkyl or arylalkyl.

In another aspect, R₁, R₄, R₅ and R₁₇ are C₁-C₄ alkyl; R₃, R₆ and R₁₈ are independently selected from C₈-C₁₈ alkyl and C₈-C₁₈ alkenyl; R₂₁, R₂₅ and R₂₆ are H; and R₁₉, R₂₀ and R₂₂ are independently selected from C₆-C₁₈ alkyl and C₆-C₁₈ alkenyl.

In another aspect, the ion pair amphiphile is prepared by reacting one or more cationic amphiphiles selected from the group consisting of 1-butyl-3-dodecyl-4.5-dihydro-3H-imidazol-1-ium chloride, hexadecyl-trimethylammonium bromide, benzyl-dodecyl-dimethylammonium chloride, dodecyl-dimethylamine, 1-butyl-4-nonyl-pyridinium bromide, dodecylamine and tributyl-hexadecylammonium bromide and one or more anionic amphiphiles selected from the group consisting of hexanoic acid, hexadecanoic acid, octadec-9-enoic acid, sulfuric acid monododecyl ester, phosphoric acid monododecyl ester, dodecanoic acid-2-hydroxy-3-phosphonooxy-propyl ester and sulfuric acid mono-(4-dodecyl-phenyl) ester.

The ion-pair amphiphiles of this invention exhibit excellent inhibition of hydrates in gas/water fluids where hydrates can form including natural gas, petroleum gas, gas condensate, crude oil, fuel oil, middle distillates, and the like. The ion-pair amphiphiles of this invention are particularly useful for preventing plugging of oil and gas transmission pipelines by hydrates. As used herein, “inhibiting” includes preventing or inhibiting the nucleation, growth and/or agglomeration of hydrate particles such that any hydrate particles are transported as a slurry in the treated fluid so that the flow of fluid through the pipeline is not sufficiently restricted as to be considered a plug.

To ensure effective inhibition of hydrates, the ion-pair amphiphiles or cationic and anionic amphiphiles should be injected prior to substantial formation of hydrates. A preferred injection point for petroleum production operations is downhole near the near the surface controlled sub-sea safety valve (SCSSV). This ensures that during a shut-in, the product is able to be disperse throughout the area where hydrates will occur. Treatment can also occur at other areas in the flowline, taking into account the density of the injected fluid. If the injection point is well above the hydrate formation depth, then the hydrate inhibitor should be formulated in a solvent with a density high enough that the inhibitor will sink in the flowline to collect at the water/oil interface. Moreover, the treatment can also be used for pipelines or anywhere in the system where there is a potential for hydrate formation.

The ion-pair amphiphile formulation or cationic and anionic amphiphile formulations are introduced into the fluid by any means suitable for ensuring dispersal of the inhibitor through the fluid being treated. Typically the inhibitor is injected using mechanical equipment such as chemical injection pumps, piping tees, injection fittings, and the like. The ion-pair amphiphile can be injected neat or in a solvent depending upon the application and requirements.

The amount of ion-pair amphiphile used to treat the fluid is the amount that effectively inhibits hydrate formation and/or aggregation. The amount of inhibitor added can be determined by one of skill in the art using known techniques such as, for example, the rocking cell test described herein. Typical doses range from about 0.05 to about 5.0 volume percent, based on the amount of the water being produced although in certain instances the dosage could exceed 5 volume percent.

The ion-pair amphiphile treatment may be used alone or in combination with thermodynamic hydrate inhibitors, kinetic hydrate inhibitors and/or anti-agglomerates as well as other treatments used in crude oil production and transport including asphaltine inhibitors, paraffin inhibitors, corrosion inhibitors, scale inhibitors, emulsion breakers and the like.

Accordingly, in an aspect, this invention further comprises treating the fluid with one or more thermodynamic hydrate inhibitors, one or more kinetic hydrate inhibitors, or one or more anti-agglomerates, or a combination thereof to the fluid.

The effective amount of thermodynamic hydrate inhibitor, kinetic hydrate inhibitor and anti-agglomerate may be empirically determined based on the characteristics of the fluid being treated, for example using the rocking cell test described herein. Typically, the ratio of thermodynamic hydrate inhibitor to ion-pair amphiphile is at least about 10:1.

In another aspect, this invention further comprises treating the fluid with one or more asphaltene inhibitors, paraffin inhibitors, corrosion inhibitors, emulsion breakers or scale inhibitors, or a combination thereof to the fluid.

The foregoing may be better understood by reference to the following examples, which are presented for purposes of illustration and are not intended to limit the scope of this invention.

### EXAMPLE 1

Rocking Cell Testing of Representative Ion-pair Amphiphiles

Representative ion-pair amphiphiles are tested under simulated field conditions corresponding to steady-state flowing, shut-in and re-start operations using the protocols and equipment described below. The fluids tested are shown in Table 1, the compositions of the fluids is shown in Tables 2 and 3 and the test conditions are shown in Table 4.

TABLE 1
Test Fluids

| Phase | Composition | Volume |
|---|---|---|
| Hydrocarbon: | Provided by producer, a GOM black oil, or | 12 mL |
| | a synthetic condensate (Table 3) | total liquid |
| Brine: | As specified to match field conditions | volume |
| Gas: | Green Canyon gas (Table 2) | |

TABLE 2
Green Canyon gas composition

| Component | mol % |
|---|---|
| Nitrogen | 0.39% |
| Methane | 87.26% |
| Ethane | 7.57% |
| Propane | 3.10% |
| iso-Butane | 0.49% |
| n-Butane | 0.79% |
| iso-Pentane | 0.20% |
| n-Pentane | 0.20% |

TABLE 3
Synthetic condensate composition

| Component | mol % | wt % |
|---|---|---|
| n-C6 | 10% | 6.66% |
| n-C7 | 12% | 9.30% |
| m-c-C6 | 8% | 6.07% |
| n-C8 | 11% | 9.71% |
| n-C9 | 7% | 6.94% |
| n-C10 | 4% | 4.40 |
| n-C12 | 3% | 3.95% |
| n-C15 | 2% | 3.28% |
| n-C19 | 2% | 4.15% |
| n-C22 | 1% | 2.40% |
| p-cymene | 25% | 25.94% |
| t-Bu-toluene | 15% | 17.19% |

TABLE 4
Test Conditions

| | |
|---|---|
| Initial charge pressure: | 2,500 psia |
| Final test pressure: | 2,000-2,200 psi |
| Initial start-up temperature: | 78° F. |
| Final test temperature: | 40° F. |
| Temperature ramp down time: | Less then 2 hours |
| Inhibitor concentration: | 0-5 vol % based on the amount of water |
| Rocking sequence: | 1. 16 hours rocking |
| | 2. 6 hours shut-in |
| | 3. 2 hours rocking |

The testing is carried out on a rocking cell apparatus as described in Dendy, Sloan E, Clathrate Hydrates of Natural Gases, Second Edition, Revised and Expanded, 1997, and Talley, Larry D. et al., “Comparison of laboratory results on hydrate induction rates in a THF rig, high-pressure rocking cell, miniloop, and large flowloop”, Annals of the New York Academy of Sciences, 2000, 314-321 According to the following protocol.
1) Add the desired amount of inhibitor to the test fluids and use the vortex mixer to thoroughly mix the fluids.
2) Fill the rocking cells with the treated test fluids at room temperature. Leave at least one cell for untreated fluids as the control blank.
3) Charge the cell with the appropriate gas. Allow time for gas to dissolve and saturate oil.
4) Begin rocking while at room temperature (outside of the hydrate envelope). Mix thoroughly for an extended period (minimum of 30 min.).
5) Cool the vessel gradually from 78° F. to 40° F. over ca. 2 hours. (Note—top off the pressure to maintain the desired pressure, or start out at a higher pessure to account for gas dissolution).
6) Start data acquisition.
7) Maintain system temperature and pressure at 40° F. and 2,000 psi pressure rocking for 16 hours (simulating steady-state flowing).
8) Stop rocking for 6 hours (simulating shut-in).
9) Resume rocking for 2 hours (simulating restart).
10) Take photos and videos at appropriate intervals or during major changes in the cells.

The cells are then evaluated and a numerical value is assigned using to the following criteria.
1: The rolling ball is stuck and/or the liquid level has dropped below an observable amount.
2: Large to medium agglomerates are present and/or the liquid level has dropped significantly and there is significant resistance to the rolling of the ball in the cell.
3: Medium agglomerates are formed in the viewable area and/or the liquid level has dropped moderately and there is some resistance to the rolling ball in the cell.
4: Small agglomerates are formed and/or the liquid level has dropped slightly, but the solution is free flowing without hindrance.
5: Tiny or no dispersed hydrates and the solution is free flowing without hindrance.

The results for representative ion-pair amphiphiles are summarized in Tables 5-8.

TABLE 5
Rocking cell test results for synthetic hydrocarbon (6° C., 10% NaCl,
2500 psi)

| Water Cut | BDCMA¹ | IPA² |
|---|---|---|
| 40 | 4.5 | 4.5 |
| 50 | 4 | 4.5 |
| 60 | 4 | 4.5 |
| 65 | 1 | 4.5 |
| 70 | 1 | 4.5 |
| 75 | 1 | 4.5 |
| 80 | 1 | 4 |
| 85 | 1 | 4 |
| 90 | 1 | 2 |

¹Benzyl dimethyl cocoamine.
²Benzyl dimethyl cocoamine/tall oil fatty acid (1:1).

TABLE 6
Rocking cell test results for GOM condensate (4° C., 15% NaCl,
2500 psi)

| Water Cut | BDCMA¹ | IPA² |
|---|---|---|
| 40 | 4.5 | 4.5 |
| 50 | 3.5 | 4.5 |
| 60 | 3 | 4.5 |
| 65 | — | 4.5 |
| 70 | 1 | 4.5 |
| 75 | — | 4.5 |
| 80 | 1 | 4 |
| 85 | — | 1 |
| 90 | 1 | 1 |

¹Benzyl dimethyl cocoamine.
²Benzyl dimethyl cocoamine/tall oil fatty acid (1:1).

TABLE 7
Rocking cell test results for GOM condensate/synthetic condensate
(1:1, 4° C., deionized water, 2500 psi, water cut 33%)

| Test | BDCMA¹ | TOFA³ | IPA² |
|---|---|---|---|
| 1 | 3 | 1 | 4.5 |
| 2 | 3.5 | 1 | 4 |
| 3 | 3 | 1⁴ | 4 |
| 4 | 4.5⁴ | 1⁴ | 4.5⁴ |

¹Benzyl dimethyl cocoamine.
²Benzyl dimethyl cocoamine/tall oil fatty acid (1:1).
³Tall oil fatty acid.
⁴Test conducted using 3.5% NaCl rather than deionized water to monitor for increased activity.

TABLE 8
Rocking cell test results for GOM black oil/synthetic condensate
(1:1, 4° C., 3.5% NaCl, 2500 psi, water cut 33%)

| Dosage | | BDCMA/Stearic acid | BDCMA/Oleic |
|---|---|---|---|
| (vol % of water) | BDCMA¹ | (1:1)¹ | acid (1:1)¹ |
| 0.50 | 3.5 | 4.5 | 4.5 |
| 0.75 | 4 | 4.5 | 4.5 |
| 1.00 | 4.5 | 4.5 | 4.5 |

¹Benzyl dimethyl cocoamine.

As shown in Tables 5-8, by replacing one half of the active ingredient, such as BDCMA, with the oppositely charged amphiphile, the activity of the formulation is increased. This effect occurs even when the oppositely charged amphiphile has shown no activity as a hydrate inhibitor itself (Table 7).

Changes can be made in the composition, operation, and arrangement of the method of the invention described herein without departing from the concept and scope of the invention as defined in the claims.

1. A method of inhibiting hydrates in a fluid comprising water, gas and optionally liquid hydrocarbon comprising treating the fluid with an effective hydrate-inhibiting amount of one or more ion-pair amphiphiles, wherein the ion-pair amphiphiles are composed of one or more cationic amphiphiles and one or more anionic amphiphiles.

2. The method of claim 1 wherein the cationic amphiphiles are selected from the group consisting of compounds of formula 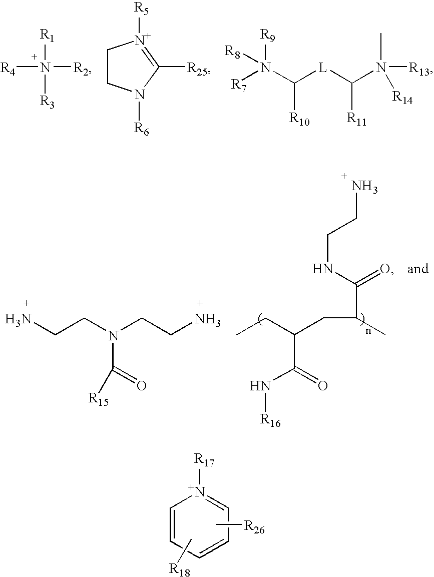
wherein R₁, R₅, R₇, R₈, R₁₂, R₁₃ and R₁₇ are independently selected from C₁-C₄ alkyl; R₂, R₉ and R₁₄ are independently selected from C₁-C₄ alkyl and arylalkyl; R₄ is C₁-C₄ alkyl, C₅-C₂₅ alkyl or C₅-C₂₅ alkenyl; R₃, R₆, R₁₀, R₁₁, R₁₅, R₁₆ and R₁₈ are independently selected from C₅-C₂₅ alkyl and C₅-C₂₅ alkenyl; R₂₅ and R₂₆ are independently selected from H, C₁-C₂₅ alkyl and C₂-C₂₅ alkenyl; L is absent, C₁-C₅ alkylene or a group of formula —CH₂CH(OH)CH₂—; and n is 1 to about 1,000.

3. The method of claim 1 wherein the cationic amphiphiles are selected from the group consisting of compounds of formula 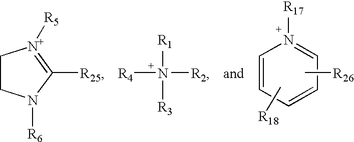
wherein R₁, R₅ and R₁₇ are independently selected from C₁-C₄ alkyl; R₂ is C₁-C₄ alkyl or arylalkyl;
R₄ is C₁-C₄ alkyl, C₅-C₂₅ alkyl or C₅-C₂₅ alkenyl; R₃, R₆ and R₁₈ are independently selected from C₅-C₂₅ alkyl and C₅-C₂₅ alkenyl, and R₂₅ and R₂₆ are independently selected from H, C₁-C₂₅ alkyl and C₂-C₂₅ alkenyl.

4. The method of claim 1 wherein the anionic amphiphile is selected from the group consisting of compounds of formula 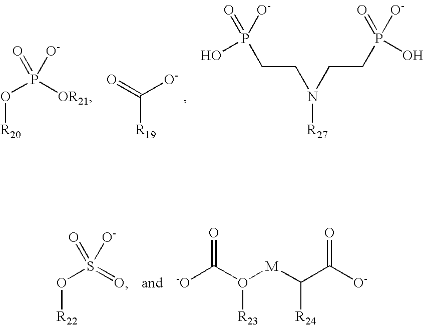
wherein R₁₉, R₂₀, R₂₂, R₂₃, R₂₇ and R₂₄ are independently selected from C₅-C₂₅ alkyl, C₅-C₂₅ alkenyl;
R₂₁ is H, C₁-C₄ alkyl or arylalkyl; and M is absent or a group of formula C₁-C₅ alkylene or a group of formula —CH₂CH(OH)CH₂—.

5. The method of claim 1 wherein the cationic amphiphiles are selected from the group consisting of compounds of formula 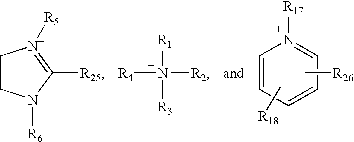
wherein R₁, R₅ and R₁₇ are independently selected from C₁-C₄ alkyl; R₂ is C₁-C₄ alkyl or arylalkyl;
R₄ is C₁-C₄ alkyl, C₅-C₂₅ alkyl or C₅-C₂₅ alkenyl; R₃, R₆ and R₁₈ are independently selected from C₅-C₂₅ alkyl and C₅-C₂₅ alkenyl, and R₂₅ and R₂₆ are independently selected from H, C₁-C₂₅ alkyl and C₂-C₂₅ alkenyl and the anionic amphiphiles are selected from the group consisting of compounds of formula 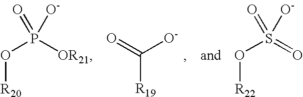
wherein R₁₉, R₂₀ and R₂₂ are independently selected from C₅-C₂₅ alkyl, C₅-C₂₅ alkenyl; and R₂₁ is H, C₁-C₄ alkyl or arylalkyl.

6. The method of claim 5 wherein R₁, R₄, R₅ and R₁₇ are C₁-C₄ alkyl; R₃, R₆ and R₁₈ are independently selected from C₈-C₁₈ alkyl and C₈-C₁₈ alkenyl; R₂₁, R₂₅ and R₂₆ are H; and R₁₉, R₂₀ and R₂₂ are independently selected from C₆-C₁₈ alkyl and C₆-C₁₈ alkenyl.

7. The method of claim 6 wherein the ion pair amphiphile is prepared by reacting one or more cationic amphiphiles selected from the group consisting of benzyl-dodecyl-dimethylammonium choride, 1-butyl-3-dodecyl-4.5-dihydro-3H-imidazol-1-ium chloride, hexadecyl-trimethylammonium bromide, dodecyl-dimethylamine, 1-butyl-4-nonyl-pyridinium bromide, dodecylamine and tributyl-hexadecylammonium bromide and one or more anionic amphiphiles selected from the group consisting of hexanoic acid, octadec-9-enoic acid, hexadecanoic acid, sulfuric acid monododecyl ester, phosphoric acid monododecyl ester, dodecanoic acid-2-hydroxy-3-phosphonooxy-propyl ester and sulfuric acid mono-(4-dodecyl-phenyl) ester.

8. The method of claim 1 wherein the ion-pair amphiphile is formulated prior to addition to the fluid.

9. The method of claim 1 wherein the treating comprises the sequential or simultaneous addition of one or more cationic amphiphiles and one or more anionic amphiphiles to the fluid.

10. The method of claim 1 further comprising adding one or more thermodynamic hydrate inhibitors, one or more kinetic hydrate inhibitors, or one or more anti-agglomerates, or a combination thereof to the fluid.

11. The method of claim 1 further comprising adding one or more asphaltine inhibitors, paraffin inhibitors, corrosion inhibitors, emulsion breakers or scale inhibitors, or a combination thereof to the fluid.

12. A composition comprising one or more ion-pair amphiphiles and one or more non aqueous solvents.

13. The composition of claim 12 wherein the non-aqueous solvents are selected from the group consisting of aromatics, alcohols, esters, aliphatics, glycols, and mixtures thereof.

14. The composition of claim 12 wherein the non-aqueous solvents are selected from the group consisting of diesel, heavy aromatic naphtha, fatty acid methyl esters, xylene, toluene, and mixtures thereof.

15. The composition of claim 12 wherein the non-aqueous solvents are selected from the group consisting of methanol, ethanol, isopropanol, 2-butoxyethanol, ethylene glycol and propylene glycol and mixtures thereof.

16. A composition comprising one or more ion-pair amphiphiles in a mixture of one or more aqueous solvents and one or more non-aqueous solvents.

17. A composition comprising one or more ion-pair amphiphiles and one or more aqueous solvents, wherein the aqueous solvents are selected from brine and seawater.
